(19) United States
(12) Patent Application Publication
(10) Pub. No.: US 2026/0017799 A1
(43) Pub. Date: Jan. 15, 2026

(54) NODULE SEGMENTATION AND RECONSTRUCTION VIA MACHINE LEARNING

(71) Applicant: Auris Health, Inc., Santa Clara, CA (US)

(72) Inventors: Dingzhong Zhang, Foster City, CA (US); Mohammad Matinfar, San Jose, CA (US)

(73) Assignee: Auris Health, Inc., Santa Clara, CA (US)

(21) Appl. No.: 19/246,065

(22) Filed: Jun. 23, 2025

Related U.S. Application Data
(60) Provisional application No. 63/671,487, filed on Jul. 15, 2024.

(51) Int. Cl. G06T 7/11; A61B 1/267; A61B 34/10; A61B 34/20; A61B 90/00; G06T 3/40; G06T 7/62; G06T 7/73; G06T 17/20

(52) U.S. Cl. CPC G06T 7/11; A61B 34/20; G06T 3/40; G06T 7/62; G06T 7/75; G06T 17/20; A61B 1/2676; A61B 2034/107; A61B 2034/2065; A61B 2090/3762; G06T 2200/04; G06T 2200/24; G06T 2207/10081; G06T 2207/10121; G06T 2207/20081; G06T 2207/20084; G06T 2207/20101; G06T 2207/20132; G06T 2207/30004

(57) ABSTRACT

This disclosure provides methods, devices, and systems for planning and performing medical procedures. The present implementations more specifically relate to analyzing objects in 3D images. In some aspects, a segmentation system may receive image data representing a 3D image of an anatomy, select a seed location for a target in the 3D image, and infer a segmentation mask associated with the seed location from at least a portion of the received image data based on a neural network model trained to segment a class of objects associated with the target. The system further extracts a polygon mesh from the segmentation mask to produce a 3D model of the target. The system can determine a spatial relationship between an instrument and the target based on a position of the 3D model relative to the 3D image. The system can also estimate a geometry of the target based on the 3D model.

### CROSS-REFERENCE TO RELATED APPLICATIONS

This application claims priority and benefit under 35 U.S.C. § 119 (e) to U.S. Provisional Patent Application No. 63/671,487, filed Jul. 15, 2024, which is incorporated herein by reference in its entirety.

### TECHNICAL FIELD

This disclosure relates generally to medical systems, and specifically to nodule segmentation and reconstructions via machine learning.

### DESCRIPTION OF RELATED ART

Many medical procedures include steps that can be performed pre-operation (also referred to as a “preoperative phase”), intra-operation (also referred to as an “intraoperative phase”), or post-operation (also referred to as a “postoperative phase”). For example, during a preoperative phase, an imaging system can be used to scan or otherwise capture images or video of an anatomy. Example suitable imaging technologies include computed tomography (CT), X-ray, fluoroscopy, positron emission tomography (PET), PET-CT, CT angiography, cone beam CT (CBCT), three-dimensional rotational angiography (3DRA), single-photon emission CT (SPECT), magnetic resonance imaging (MRI), optical coherence tomography (OCT), and ultrasound, among other examples. The images can be used, during an intraoperative phase, to help guide or navigate a medical instrument to a nodule (also referred to as a “treatment site”) within the anatomy. For example, the position of the nodule can be determined from the images using image segmentation techniques while the position and orientation of the instrument can be tracked in real-time via sensors (such as electromagnetic sensors) disposed on the instrument. The spatial relationship between the instrument and the nodule can be determined by mapping the nodule position to a coordinate space associated with the sensors or by mapping the instrument position (and orientation) to a coordinate space associated with the image. Thus, the accuracy of the determined spatial relationship depends, at least in part, on the accuracy of the image segmentation.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a method for analyzing a target within an anatomy. The method includes steps of receiving image data representing a three-dimensional (3D) image of the anatomy; selecting a seed location for the target in the 3D image of the anatomy; inferring a segmentation mask associated with the seed location from at least a portion of the received image data based on a neural network model trained to segment a class of objects associated with the target; and generating a polygon mesh representing a geometry of the target based on the segmentation mask.

Another innovative aspect of the subject matter of this disclosure can be implemented in a controller for a medical system, including a processing system and a memory. The memory stores instructions that, when executed by the processing system, cause the controller to receive image data representing a 3D image of the anatomy; select a seed location for a target in the 3D image of the anatomy; infer a segmentation mask associated with the seed location from at least a portion of the received image data based on a neural network model trained to segment a class of objects associated with the target; and generate a polygon mesh representing a geometry of the target based on the segmentation mask.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows example components of the control system and the robotic system of FIG. 1, according to some implementations.

FIG. 3 shows a block diagram of an example localization system, according to some implementations.

FIG. 4 shows a block diagram of an example segmentation system, according to some implementations.

FIG. 5 shows a block diagram of an example machine learning system, according to some implementations.

FIG. 6 shows a block diagram of an example target reconstruction system, according to some implementations.

FIG. 7 shows a block diagram of an example image cropping system, according to some implementations.

FIG. 8 shows an example image of an anatomy with volume of interest (VOI) padding, according to some implementations.

FIG. 9 shows an example graphical interface depicting a reconstructed nodule superimposed on images of an anatomy, according to some implementations.

FIG. 10 shows a block diagram of an example controller for a medical system, according to some implementations.

FIG. 11 shows an illustrative flowchart depicting an example operation for analyzing a target within an anatomy, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, many medical procedures include a preoperative phase that precedes an intraoperative phase. During the preoperative phase, for some medical procedures, an imaging system may be used to scan or otherwise capture images or video of at least a portion of an anatomy. For example, a computed tomography (CT) scanner can be used to acquire tomographic images (also referred to as “tomograms” or “CT scans”) of a patient's lungs during the preoperative phase for a bronchoscopy. A tomogram is a cross-section or slice of a three-dimensional (3D) volume. For example, multiple tomograms can be stacked or combined to recreate the 3D volume (such as a 3D image of the lungs). Thus, tomograms can be used to detect a precise location or position (in 3D space) of a nodule or target in the lungs. During the intraoperative phase, for some medical procedures, a medical system can use the preoperative images to generate a graphical interface for navigating a medical instrument within the anatomy. For example, during a bronchoscopy, the medical system can detect a pose of an endoscope (such as a position and orientation of the scope in 3D space) based on sensor data received via an electromagnetic (EM) sensor disposed on the tip of the scope and map the pose of the endoscope to a 3D image of the lungs depicted by the tomograms.

Accordingly, the graphical interface may depict a spatial relationship between the medical instrument and the target within the anatomy based on the sensor data and the image data. The image data and the sensor data are often associated with different coordinate spaces. Thus, the medical system may “register” the image space with the sensor space to facilitate real-time navigation. As used herein, the term “registration” refers to a mapping or transformation between different coordinate spaces. For example, a medical system can register an imaging system used for capturing images of an anatomy (such as a CT scanner) with a sensor system used for tracking a pose of a medical instrument within the anatomy (such as an EM field generator) by determining a mapping or spatial transformation that maps any point or vector in the image space to a respective point or vector in the sensor space (such as a transformation matrix). The terms “mapping,” “transformation,” “spatial transformation,” and “registration matrix,” may be used interchangeably herein. The terms “respective” and “corresponding” also may be used interchangeably herein.

To provide more detailed navigational guidance, the medical system may segment the image data to determine the position of the target in a coordinate space associated with the images (also referred to as the “image space”). Image segmentation refers to various techniques for partitioning a digital image into groups of voxels having related characteristics and/or features. The medical system may determine the spatial relationship between the instrument and the target by mapping the target position from the image space to a coordinate space associated with the sensor data (also referred to as the “sensor space”) or by mapping the instrument pose from the sensor space to the image space. The accuracy of the determined spatial relationship thus depends, at least in part, on the accuracy of the image segmentation. Some existing medical systems perform image segmentation using thresholding techniques (such as by classifying voxels having values higher than a threshold intensity as belonging to the target). However, thresholding often provides a relatively coarse approximation of a target's edges or boundaries (often resulting in over-segmentation or under-segmentation).

Aspects of the present disclosure recognize that image segmentation can be more accurately performed through machine learning. Machine learning is a technique for improving the ability of a computer system to perform a certain task. Machine learning generally comprises a training phase and an inferencing phase. During the training phase, a machine learning system is provided with one or more “answers” and a large volume of raw training data associated with the answers. The machine learning system analyzes the training data to learn a set of rules that can be used to describe each of the answers. During the inferencing phase, the machine learning system may infer answers from new data using the learned set of rules. Deep learning is a form of machine learning in which the inferencing and training phases are performed over multiple layers. Deep learning architectures are often referred to as “artificial neural networks” due to the manner in which information is processed (similar to a biological nervous system). For example, each layer of a neural network may be composed of one or more “neurons” that perform a different transformation on the output data from a preceding layer so that the final output of the neural network results in the desired inferences. The set of transformations associated with the various layers of the network is referred to as a “neural network model.”

Various aspects relate generally to systems and techniques for identifying objects of interest (such as nodules) within an anatomy, and more particularly, to image segmentation techniques that use machine learning to produce 3D models of such objects of interest. In some aspects, a segmentation system may receive image data representing a 3D image of an anatomy, select a seed location for an object of interest (also referred to herein as a “target”) in the 3D image, and infer a segmentation mask associated with the seed location from at least a portion of the received image data based on a neural network model trained to segment a class of objects associated with the target. In some implementations, the segmentation system may receive user input indicating the seed location. In some other implementations, the segmentation system may determine the seed location based on one or more image processing operations (such as through machine learning or statistical analysis). In some implementations, the segmentation mask may be a binary mask that classifies each voxel of the 3D image as belonging to the target or a background object. In some implementations, the segmentation system may further extract a polygon mesh from the segmentation mask to produce a 3D model of the target that can be superimposed or projected onto the 3D image of the anatomy. In some aspects, the segmentation system may determine a spatial relationship between an instrument and the target based on a position of the 3D model relative to the 3D image. In some other aspects, the segmentation system may estimate a geometry (such as a volume or diameter) of the target based on a geometry of the 3D model.

Particular implementations of the subject matter described in this disclosure can be implemented to realize one or more of the following potential advantages. By inferring a segmentation mask using a neural network model, aspects of the present disclosure can produce a 3D model that more accurately depicts the geometry and dimensions of a target within an anatomy compared to existing image thresholding techniques. Accordingly, the 3D model of the present implementations can provide more precise and accurate information regarding the target. For example, the 3D model can be used to determine a more precise and accurate spatial relationship between the target and a medical instrument (such as an endoscope) for navigating the instrument to the target. Because the edges or boundaries of the 3D model more accurately reflect the actual edges or boundaries of the target, the 3D model can also be used to confirm tool-in-lesion of a medical instrument (such as a biopsy needle). In addition to improving the accuracy of intraoperative procedures, the 3D model of the present implementations can also improve the accuracy of postoperative procedures and/or other treatments or procedures that rely on monitoring a nodule's geometric and mechanical properties and/or location (such as for planning drug dosage and/or delivery locations, biopsy patterns and/or locations, and ablation, among other examples).

Although certain aspects of the present disclosure are described in detail herein in the context of bronchoscopy, it should be understood that the systems and techniques of the present disclosure may be applicable to any medical procedure. Example medical procedures may include therapeutic procedures, diagnostic procedures, percutaneous procedures, and non-percutaneous procedures, among other examples. Example endoscopic procedures include bronchoscopy, ureteroscopy, gastroscopy, nephroscopy, and nephrolithotomy, among other examples. The terms “scope,” “endoscope,” “catheter,” and “instrument” may be used interchangeably herein. The systems and techniques of the present disclosure also may be applicable to procedures and/or treatments that do not involve navigating a medical instrument within an anatomy (such as for planning drug dosage and/or delivery locations, biopsy patterns and/or locations, or ablation, among other examples).

Aspects of the present disclosure may be used to perform robotic-assisted medical procedures, such as endoscopic access, percutaneous access, or treatment for a target anatomical site. For example, robotic tools may engage or control one or more medical instruments (such as an endoscope) to access a target site within an anatomy or perform a treatment at the target site. In some implementations, the robotic tools may be guided or controlled by a physician. In some other implementations, the robotic tools may operate in an autonomous or semi-autonomous manner. Although systems and techniques are described herein in the context of robotic-assisted medical procedures, the systems and techniques may be applicable to other types of medical procedures (such as procedures that do not rely on robotic tools or only utilize robotic tools in a very limited capacity). For example, the systems and techniques described herein may be applicable to medical procedures that rely on manually operated medical instruments (such as an endoscope that is exclusively controlled and operated by a physician). The systems and techniques described herein also may be applicable beyond the context of medical procedures (such as in simulated environments or laboratory settings, such as with models or simulators, among other examples)

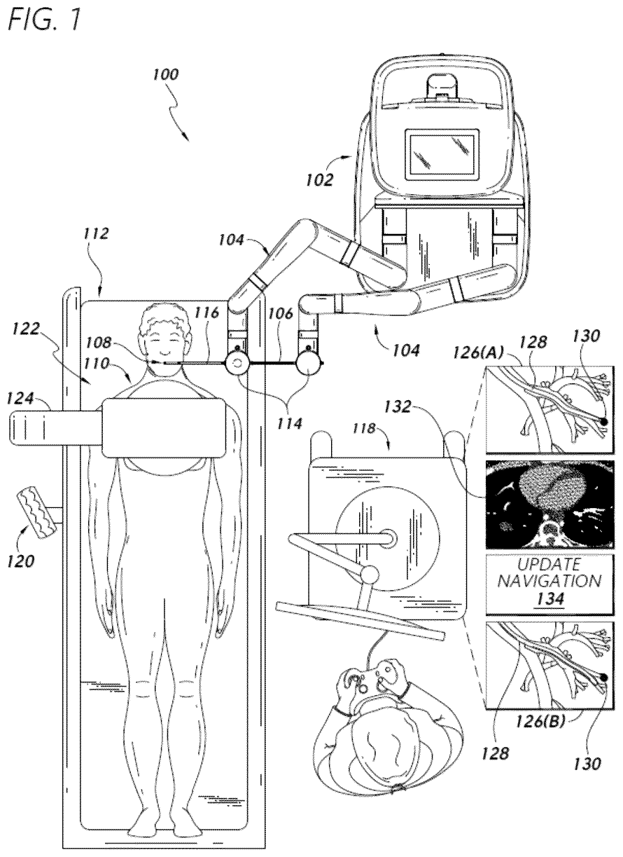

FIG. 1 shows an example medical system 100 (also referred to as a “surgical medical system” or a “robotic medical system”), according to some implementations. As shown in FIG. 1, the medical system 100 may be arranged for diagnostic or therapeutic bronchoscopy. The medical system 100 can include and utilize a robotic system 102 which can be implemented, for example, as a robotic cart. Although the medical system 100 is shown as including various cart-based systems or devices, the concepts disclosed herein can be implemented in any type of robotic system or arrangement, such as robotic systems employing rail-based components, table-based robotic end-effectors, or manipulators, among other examples. The robotic system 102 may include one or more robotic arms 104 (also referred to as “robotic positioners”) configured to position or otherwise manipulate a medical instrument 106 (such as a steerable endoscope or another elongate instrument). For example, the medical instrument 106 can be advanced through a natural orifice access point (such as the mouth 108 of a patient 110 positioned on a table 112) to deliver diagnostic or therapeutic treatment. Although described in the context of a bronchoscopy procedure, the medical system 100 also may be used to perform other types of medical procedures. Example suitable procedures include gastro-intestinal (GI) procedures, renal procedures, urological procedures, and nephrological procedures, among other examples.

With the robotic system 102 properly positioned, the medical instrument 106 can be inserted into the patient 110 robotically, manually, or a combination thereof. For example, the one or more robotic arms 104, or instrument drivers 114 coupled thereto, can control the medical instrument 106. In some implementations, the medical instrument 106 may be advanced within a sheath 116. For example, the sheath 116 may be coupled to, or controlled by, a robotic arm 104. In some implementations, the medical instrument 106 and the sheath 116 may each be coupled to a respective instrument driver from a set of instrument drivers 114. The instrument drivers 114 can be repositionable in space by manipulating the one or more robotic arms 104 into different angles or positions.

In the example of FIG. 1, the medical instrument 106 can be directed down the patient's trachea and lungs after insertion or advanced to a target destination or operative site. In some implementations, to enhance navigation through the patient's lung network or reach the desired target, the medical instrument 106 may be manipulated to telescopically extend from the outer sheath 116 to obtain enhanced articulation or greater bend radius. The use of separate instrument drivers 114 can allow the medical instrument 106 and sheath 116 to be driven independently of each other.

In some implementations, the medical instrument 106 may include an elongate member or shaft configured to be inserted or retracted, articulated, or otherwise moved within the anatomy. Further, in some implementations, the medical instrument 106 may include one or more imaging devices (such as cameras) positioned on a distal end of the elongate shaft or deployed through a working channel of the elongate shaft. The imaging devices can be configured to generate or capture image (or video) data or send the image data to another device or component. In some implementations, the medical instrument 106 may include an instrument base or one or more handles positioned at a proximal end of the medical instrument 106. The instrument base can be coupled to a manipulator (such as an end of a robotic arm 104). The instrument base can include one or more drive inputs coupled to one or more drive outputs of the manipulator, wherein the drive inputs or drive outputs act as an interface.

In some implementations, the medical instrument 106 may include a working channel configured to receive one or more other instruments or elements therein or provide other functionality. The working channel can extend axially, such as along the length of the medical instrument 106. Furthermore, the medical instrument 106 can include or be associated with one or more elongate movement members (such as pulls wires) that can extend from a proximal end through the elongate shaft to the distal end of the elongate shaft. The elongate movement members can be manipulated, such as by manipulators on the one or more robotic arms 104, to control actuation of the elongate movement members.

In some implementations, the medical instrument 106 may include one or more sensors, such as electromagnetic (EM) sensors, shape sensors (such as shape sensing fiber), accelerometers, gyroscopes, satellite-based positioning sensors (such as global positioning system (GPS) sensors), or radio-frequency (RF) transceivers, among other examples. The sensors can be configured to generate or produce sensor data or provide the sensor data to another device or component. The sensors can be disposed at a distal end of the elongate shaft or along a length of the elongate shaft. In some implementations, the medical instrument 106 may be configured to receive an elongate member or device through a working channel, wherein the elongate member includes one or more sensors along a length of the elongate member. One or more sensors on the medical instrument 106 may provide sensor data to control circuitry of the medical system 100, which is then used to determine a position, orientation, or shape of the medical instrument 106.

The medical system 100 can also include a control system 118 (also referred to as a “control tower” or “mobile tower”). The control system 118 can be communicatively coupled (such as via wired or wireless connections) to the robotic system 102 to control various aspects of the robotic system 102 (such as electronics, optics, sensors, or power) or one or more subsystems associated with the robotic system 102, such as a fluid management system (not shown). Placing such functionality in the control system 118 can allow for a smaller form factor of the robotic system 102 that may be more easily adjusted or re-positioned by an operator or user. Additionally, the division of functionality between the robotic system 102 and the control system 118 can reduce operating room clutter and facilitate efficient clinical workflow.

The medical system 100 can include an electromagnetic (EM) field generator 120, which is configured to broadcast or emit an EM field that can be detected by various EM sensors, such as a sensor disposed on the medical instrument 106. The EM field can induce small electric currents in coils of the EM sensors, which can be analyzed to determine a position, angle, or orientation of the EM sensors relative to the EM field generator 120. Although EM fields and EM sensors are described in many examples herein, position sensing systems or sensors can include various other types of position sensing systems or sensors, such as optical position sensing systems or sensors, image-based position sensing systems or sensors, among other examples.

The medical system 100 can further include an imaging system 122 (also referred to as an “imaging device”) configured to generate, provide, or send image data (also referred to as “images”) to another device or system. For example, the imaging system 122 can generate image data depicting an anatomy of the patient 110 and provide the image data to the control system 118, the robotic system 102, or another device. The imaging system 122 may include an emitter or energy source (such as an X-ray source) or a detector (such as an X-ray detector) mounted on a C-shaped arm support 124, which allows for flexibility in positioning around the patient 110 to capture images from various angles without moving the patient 110. Use of the imaging system 122 can provide visualization of internal structures or anatomy, which can be used for a variety of purposes, including navigation of the medical instrument 106 (such as by providing images of internal anatomy to a user) and localization of the medical instrument 106 (based on an analysis of image data), among other examples. In some aspects, the imaging system 122 may enhance the efficacy or safety of a medical procedure, such as a bronchoscopy, by providing clear, continuous visual feedback to the operating surgeon or team.

In some implementations, the imaging system 122 may be a mobile device configured to move around an environment. For example, the imaging system 122 can be positioned next to the patient 110 (as shown in FIG. 1) during a particular phase of a procedure and removed when the imaging system 122 is no longer needed. In some other implementations, the imaging system 122 may be part of the table 112 or other equipment in an operating environment. The imaging system 122 can be implemented as a Computed Tomography (CT) machine or system, X-ray machine or system, fluoroscopy machine or system, Positron Emission Tomography (PET) machine or system, PET-CT machine or system, CT angiography machine or system, Cone-Beam CT (CBCT) machine or system, three-dimensional rotational angiography (3DRA) machine or system, single-photon emission computed tomography (SPECT) machine or system, Magnetic Resonance Imaging (MRI) machine or system, Optical Coherence Tomography (OCT) machine or system, or ultrasound machine or system, among other examples. In some implementations, the medical system 100 may include different types of imaging systems that can be used or positioned over the patient 110 during different phases or portions of a procedure depending on the needs at that time.

In some implementations, the imaging system 122 may be configured to process multiple images (also referred to as “image data”) to generate a three-dimensional (3D) view or model. For example, the imaging device 122 can be implemented as a CT machine configured to capture or generate a series of images (also referred to as “tomograms) or image data representing two-dimensional (2D) cross-sections or slices of a 3D volume from different angles around the patient 110, and then use one or more algorithms to reconstruct these images or image data into a 3D model. The 3D model can be provided to the control system 118, robotic system 102, or another device, such as for processing or display.

In some implementations, image data from the imaging system 122 may be used to localize various elements, such as the medical instrument 106, a target within the anatomy, or specific anatomical features, among other examples. As used herein, the terms “localize,” “localization,” or “localizing” refer to any processes for determining or estimating a position (or location) and/or orientation (or heading), collectively referred to as the “pose,” of the instrument or the target (or any other element) within a given space or environment. For example, the control system 118 can be configured to provide navigation information during a procedure to assist a user navigating the medical instrument 106 within the anatomy to reach a target (such as a desired treatment site or location). In some implementations, a target can include a nodule, such as in the context of certain bronchoscopy procedures. To illustrate, the control system 118 can display a navigation view or graphical data 126 that includes an instrument indicator 128 representing the medical instrument 106, a target indicator 130 representing the target, and an anatomical map. The navigation data 126(A) (such as initial navigation data) can be determined based on sensor data from a sensor of the medical instrument 106 (such as EM sensor data associated with the EM field generator 120), a map of the anatomy, or a location of the target. In some implementations, the map or location of the target may be determined based on preoperative data, such as data obtained during a preoperative procedure to find a target location or map the anatomy.

In some implementations, the navigation data 126(A) may be dynamically updated based on image data 132 from the imaging system 122. For example, the control system 118 can receive the image data 132 and analyze the image data 132 to determine a current or actual spatial relationship between the medical instrument 106 and the target. In some implementations, the control system 118 may display the image data 132 to a user, receive user input indicating a position of the medical instrument 106 or a position of the target in the image data 132, and analyze the image data 132 based on the user input to determine the current spatial relationship. If the control system 118 determines that the navigation data 126(A) incorrectly depicts the location of the medical instrument 106 (such as where the spatial relationship associated with the image data 132 is different than the spatial relationship associated with the navigation data 126(A)), the control system 118 may update the navigation data 126(A) at 134 and provide updated navigation data 126(B) that reflects the current or near real-time position of the medical instrument 106 relative to the target or the map.

The various components of the medical system 100 can be communicatively coupled to each other over a network, which can include a wireless or wired network. Example networks include one or more personal area networks (PANs), local area networks (LANs), wide area networks (WANs), Internet area networks (IANs), cellular networks, the Internet, personal area networks (PANs), body area network (BANs), etc. In some examples, various communication interfaces can include wireless technology, such as Bluetooth, Wi-Fi, near-field communication (NFC), or the like. Furthermore, in some examples, the various components of the medical system 100 can be connected for data communication, fluid exchange, power exchange, and so on, via one or more support cables, tubes, connections, or the like.

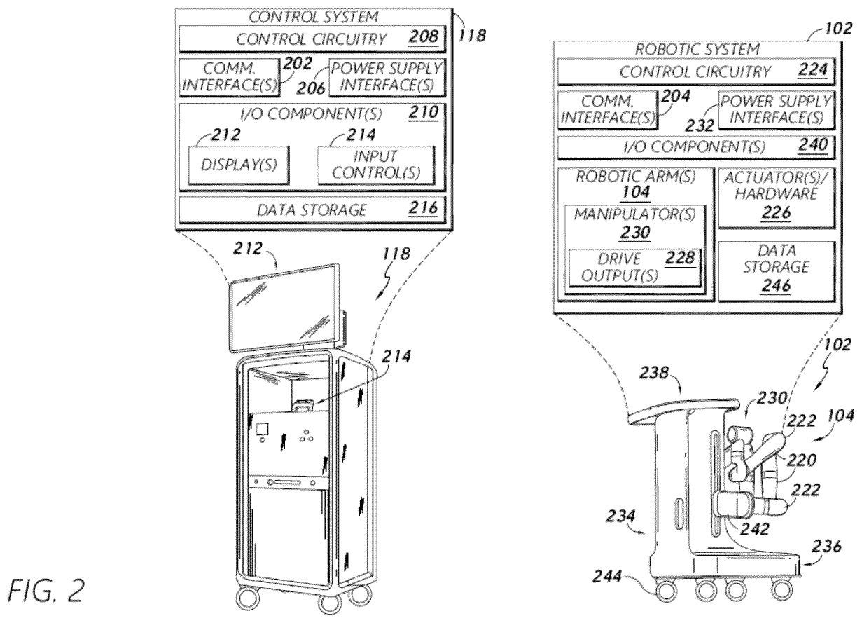

FIG. 2 shows example components of the control system 118 and the robotic system 102 of FIG. 1, according to some implementations. In the examples of FIG. 2, the control system 118 and the robotic system 102 are implemented as a tower and a robotic cart, respectively. However, the control system 118 and robotic system 102 can be implemented in other manners. The control system 118 can be coupled to the robotic system 102 and operate in cooperation therewith to perform a medical procedure. For example, the control system 118 can include communication interface(s) 202 for communicating with communication interface(s) 204 of the robotic system 102 via a wireless or wired connection (such as to control the robotic system 102). In some implementations, the control system 118 may communicate with the robotic system 102 to receive position or sensor data therefrom relating to the position of sensors associated with an instrument or member controlled by the robotic system 102. For example, the control system 118 may communicate with the EM field generator 120 to control generation of an EM field in an area around a patient. The control system 118 can further include one or more power supply interface(s) 206.

The control system 118 can include control circuitry 208 configured to cause one or more components of the medical system 100 to actuate or otherwise control any of the various system components, such as carriages, mounts, arms or positioners, medical instruments, imaging devices, position sensing devices, or sensors, among other examples. Further, the control circuitry 208 can be configured to perform other functions, such as cause display of information, process data, receive input, communicate with other components or devices, or any other function or operation described herein.

The control system 118 can further include one or more input or out (I/O) components 210 configured to assist a physician or others in performing a medical procedure. For example, the one or more I/O components 210 can be configured to receive input or provide output to enable a user to control or navigate the medical instrument 106, the robotic system 102, or other instruments or devices associated with the medical system 100. The control system 118 can include one or more displays 212 to provide, display or otherwise present various information regarding a procedure. For example, the one or more displays 212 can be used to present navigation information including a virtual anatomical model of anatomy with a virtual representation of a medical instrument, image data, or other information. The one or more I/O components 210 can include one or more user input control(s) 214, which can include any type of user input (or output) devices or device interfaces, such as one or more buttons, keys, joysticks, handheld controllers (such as video-game-type controllers), computer mice, trackpads, trackballs, control pads, sensors (such as motion sensors or cameras) that capture hand gestures and finger gestures, touchscreens, toggle (such as button) inputs, or interfaces or connectors therefore. In some implementations, such inputs can be used to generate commands for controlling one or more medical instruments, robotic arms, or other components.

The control system 118 can also include data storage 216 configured to store executable instruments (such as computer-readable instructions) that can be executed by the control circuitry 208 to cause the control circuitry 208 to perform various operations or functionality described herein. In some implementations, the data storage 216 also may store telemetry or runtime data (such as sensor data or image data) generated by the medical system 100 or otherwise captured or acquired during a medical procedure. In some implementations, two or more components of the control system 118 can be electrically or communicatively coupled to each other.

The robotic system 102 can include the one or more robotic arms 104 configured to engage with or control, for example, the medical instrument 106 or other elements or components to perform one or more aspects of a procedure. As shown in FIG. 2, each robotic arm 104 can include multiple segments 220 coupled to joints 222, which can provide multiple degrees of movement or freedom. The robotic system 102 can be configured to receive control signals from the control system 118 to perform certain operations, such as to position one or more of the robotic arms 104 in a particular manner or manipulate an instrument, among other examples. In response, the robotic system 102 can control, using control circuitry 224 thereof, actuators 226 or other components of the robotic system 102 to perform the operations. For example, the control circuitry 224 can control insertion or retraction, articulation, or roll of a shaft of the medical instrument 106 or other instrument by actuating one or more drive outputs 228 of a manipulator 230 (or end-effector) coupled to a base of a robotically-controllable instrument. The drive outputs 228 can be coupled to a drive input on an associated instrument, such as an instrument base of an instrument that is coupled to the associated robotic arm 104. The robotic system 102 also may include one or more power supply interfaces 232.

The robotic system 102 can include a support column 234, a base 236, or a console 238. The console 238 can provide one or more I/O components 240, such as a user interface for receiving user input or a display screen (or a dual-purpose device, such as a touchscreen) to provide the physician or user with preoperative or intraoperative data. The support column 234 can include an arm support 242 (also referred to as a “carriage”) for supporting the deployment of the one or more robotic arms 104. The arm support 242 can be configured to vertically translate along the support column 234. Vertical translation of the arm support 242 allows the robotic system 102 to adjust the reach of the robotic arms 104 to meet a variety of table heights, patient sizes, or physician preferences. The base 236 can include wheel-shaped casters 244 (also referred to as “wheels”) that allow the robotic system 102 to move around the operating room. After reaching the appropriate position, the casters 244 can be immobilized using wheel locks to hold the robotic system 102 in place during the procedure.

The joints 222 of each robotic arm 104 can each be independently-controllable or provide an independent degree of freedom available for instrument navigation. In some implementations, each robotic arm 104 may include seven joints that provide seven degrees of freedom, including “redundant” degrees of freedom. Redundant degrees of freedom can allow robotic arms 104 to be controlled to position their respective manipulators 230 at a specific position, orientation, or trajectory in space using different linkage positions and joint angles. This allows for the robotic system 102 to position or direct a medical instrument from a desired point in space while allowing the physician to move the joints 222 into a clinically advantageous position away from the patient to create greater access, while avoiding collisions.

The one or more manipulators 230 (or end-effectors) can be coupled to an instrument base or handle, which can be attached using a sterile adapter component. The combination of the manipulator 230 and instrument base, as well as any intervening mechanics or couplings (such as the sterile adapter), can be collectively referred to as the manipulator or a manipulator assembly. Manipulators or manipulator assemblies can provide power or control interfaces. Example interfaces may include connectors to transfer pneumatic pressure, electrical power, electrical signals, or optical signals from the robotic arm 104 to an instrument base. Manipulators or manipulator assemblies can be configured to manipulate medical instruments (such as surgical tools) using techniques including, for example, direct drives, harmonic drives, geared drives, belts or pulleys, or magnetic drives, among other examples.

The robotic system 102 can also include data storage 246 configured to store executable instruments (such as computer-readable instructions) that can be executed by the control circuitry 224 to cause the control circuitry 224 to perform various operations or functionality described herein. In some implementations, the data storage 216 also may store telemetry or runtime data (such as sensor data or image data) generated by the medical system 100 or otherwise captured or acquired during a medical procedure. In some implementations, two or more of the components of the robotic system 102 can be electrically or communicatively coupled to each other.

Data storage (including the data storage 216, data storage 246, or other data storage or memory) can include any suitable or desirable type of computer-readable media. For example, computer-readable media can include one or more volatile data storage devices, non-volatile data storage devices, removable data storage devices, or nonremovable data storage devices implemented using any technology, layout, or data structure(s) or protocol, including any suitable or desirable computer-readable instructions, data structures, program modules, or other types of data.

Computer-readable media that can include, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

Functionality described herein can be implemented by the control circuitry 208 of the control system 118 or the control circuitry 224 of the robotic system 102, such as by the control circuitry 208 or 224 executing instructions to cause the control circuitry 208 or 224 to perform the functionality. Control circuitry (including the control circuitry 208, control circuitry 224, or other control circuitry) can include circuitry embodied in a robotic system, control system or tower, instrument, or any other component or device. Control circuitry can include any collection of processors, processing circuitry, processing modules or units, chips, dies (such as semiconductor dies including one or more active or passive devices or connectivity circuitry), microprocessors, micro-controllers, digital signal processors, microcomputers, central processing units, field-programmable gate arrays, programmable logic devices, state machines (such as hardware state machines), logic circuitry, analog circuitry, digital circuitry, or any device that manipulates signals (analog or digital) based on hard coding of the circuitry or operational instructions.

Control circuitry referenced herein can further include one or more circuit substrates (such as printed circuit boards), conductive traces and vias, or mounting pads, connectors, or components. Control circuitry can further include one or more storage devices, which may be embodied in a single device, a plurality of devices, or embedded circuitry of a device. Such data storage can comprise read-only memory, random access memory, volatile memory, non-volatile memory, static memory, dynamic memory, flash memory, cache memory, data storage registers, or any device that stores digital information. In examples in which control circuitry includes a hardware or software state machine, analog circuitry, digital circuitry, or logic circuitry, data storage device(s) or register(s) storing any associated operational instructions can be embedded within, or external to, the circuitry comprising the state machine, analog circuitry, digital circuitry, or logic circuitry.

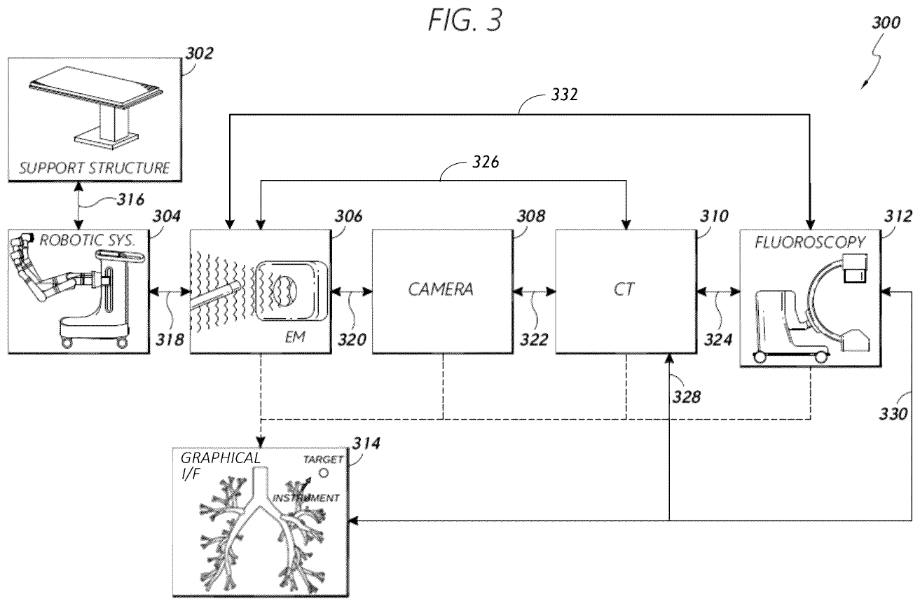

FIG. 3 shows a block diagram of an example localization system 300, according to some implementations. The localization system 300 includes various positioning or imaging systems or modalities 302-312 (also referred to as “subsystems”), which can be implemented to facilitate anatomical mapping, navigation, positioning, or visualization for procedures in accordance with one or more examples. For example, the various systems 302-312 can be configured to provide data for generating an anatomical map, determining a location of an instrument, determining a location of a target, or performing other techniques.

Each of the systems 302-312 can be associated with a respective coordinate space (also referred to as a “position coordinate frame”) or can provide data or information relating to instrument or anatomy locations, wherein registering the various coordinate spaces to one another can allow for integration of the various systems to provide mapping, navigation, or instrument visualization. For example, registering a first modality to a second modality can allow for determined positions in the first modality to be tracked or superimposed on or in a reference frame associated with the second modality, thereby providing layers of positional information that can be combined to provide a robust localization system.

In some aspects, the system 300 may be configured to perform one or more localization or localizing techniques. In some implementations, the anatomical space in which a medical instrument can be localized (such as where a pose or shape of the instrument is determined or estimated) may be a 2D or 3D portion of a patient's tracheobronchial airways, vasculature, urinary tract, gastrointestinal tract, or any organ or space accessed via lumens. Various modalities can be implemented to provide images, representations, or models of the anatomical space. For example, an imaging modality can be implemented, which can include, for example, X-ray, fluoroscopy, CT, PET, PET-CT, CT angiography, CBCT, 3DRA, SPECT, MRI, OCT, or ultrasound, among other examples. In some implementations, the imaging modality may be used to capture or acquire images of a patient's anatomy during a preoperative phase of a medical procedure. In some other implementations, the imaging modality may be used to capture or acquire images of a patient's anatomy during an intraoperative phase of the medical procedure.

The systems 302-312 can provide information for generating a graphical interface (I/F) 314 that includes navigation information for navigating an instrument to a target within an anatomy (such as the navigation data 126(A) or 126(B) of FIG. 1). For example, the navigation information may include an anatomical map, an estimated position, orientation, and/or shape of the instrument. The navigation information also may include a shape, boundary, eccentricity, texture, and/or position of the target. In some implementations, the graphical user interface 314 or other localization information may be displayed to a user, such as a physician, during a medical procedure to assist the user in performing the procedure. For example, a visualization of a tracked instrument can be superimposed on an anatomical map depicted by the graphical user interface 314 based on position or sensor data associated with the tracked medical instrument.

As shown in FIG. 3, the system 300 can include a support structure 302 (such as a surgical bed or other patient positioning or support platform). For example, the support structure 302 includes a planar surface that contacts and supports the patient. In some implementations, the position of the support structure 302 may be known based on data maintained relating to the position of the support structure 302 within the surgical or procedure environment. In some other implementations, the position of the support structure 302 may be sensed or otherwise determined using one or more markers or an appropriate imaging or positioning modality.

The system 300 can further include a robotic system 304 (such as a robotic cart or other device or system including one or more robotic end effectors). In some implementations, the robotic system 304 may be one example of the robotic system 102 of FIGS. 1 and 2. Data relating to the position or state of robotic arms, actuators, or other components of the robotic system 304 can be known or derived from robotic command data or other robotic data relative to a coordinate frame of the robotic system 304. In some examples, reference frame registration 316 occurs between the support structure 302 and the robotic system 304, which can be a relatively coarse registration, in some implementations, based on robotic system or cart-set-up procedure (which can have any suitable or desirable scheme).

The system 300 can further include an electromagnetic (EM) sensor system 306, which can include an EM field generator (such as the EM field generator 120 of FIG. 1) and one or more EM sensors. An EM sensor can be associated with a portion of an instrument that is tracked or controlled, such as a distal end (or tip) of the instrument or along a length of the instrument or other elongate member (such as a working channel) disposed in a lumen of the instrument. In some implementations, the EM field generator can be mechanically coupled to the support structure 302 or the robotic system 304 such that registration or association 318 between such systems can be known or determined. In some implementations, the registration 318 between the EM sensor system 306 and the robotic system 304 can be determined through forward kinematics or field generator mount transform information. For example, the field generator can be mounted to the support structure 302 such that the position of the field generator can be known relative to the robotic system positioning frame based on a known relationship between the position of the support structure 302 and the robotic system 304. The EM sensor system 306 can provide instrument pose or path information based on sensor readings associated with the instrument.

The system 300 can further include an optical camera system 308 including one or more cameras or other imaging devices configured to generate images of patient anatomy within a visual field thereof (such as real-time image data) during a surgical procedure. In some implementations, registration 320 between the optical camera system 308 and the EM sensor system 306 can be achieved through identification of features having EM sensor data associated therewith, such as a medical instrument tip, in images generated by the optical camera system 308. The registration 320 can further be based at least in part on hand-eye interaction of the physician when viewing real-time camera images while the EM-sensor-equipped endoscope is navigating in the patient anatomy.

The system 300 can further include a computed tomography (CT) imaging system 310 configured to generate CT images of the patient anatomy, which can be performed preoperatively or intraoperatively. The CT imaging system 310 is generally used for scanning a relatively large volume. In some implementations, image processing can be implemented for registration 322 of the CT image data with the camera image data generated by the optical camera system 308. For example, common features identified in both camera image data and CT image data can be identified to relate the CT image frame to the camera image frame in space. In some examples, the CT imaging system 310 can be used to generate preoperative imaging data for producing the graphical user interface 314 or for path navigation planning.

In some aspects, the CT imaging system 310 may be registered 326 to the EM sensor system 306 through various techniques. In some implementations, a mechanical structure of the CT imaging system 310 can have a known physical transform or relationship with respect to a mounting position of the EM field generator of the EM sensor system 306. Such known relationship can be used to register the CT image space to the EM sensor space. The connection 328 represents a mapping or relationship between the CT imaging system 310 and an anatomical map depicted by a graphical user interface 314.

The system 300 can further include a fluoroscopy imaging system 312 configured to generate tomographic images (such as real-time X-ray images) of the surgical site. The fluoroscopy imaging system 312 is generally used for scanning a smaller volume compared to the CT imaging system 310. In some implementations, the fluoroscopy imaging system 312 may be one example of the imaging system 122 of FIG. 1. For example, the fluoroscopy imaging system 312 may include a CBCT scanner coupled to a C-arm. In some implementations, the fluoroscopy imaging system 312 may be used with a contrast agent introduced into the anatomy to generate image data representing patient anatomy or instrumentation. In some implementations, the fluoroscopy imaging system 312 may be registered 324 to the CT imaging system 310 using any image processing technique suitable for such registration.

In some aspects, the fluoroscopy imaging system 312 may be registered 332 to the EM sensor system 306 through various techniques. In some implementations, a mechanical structure of the fluoroscopy imaging system 312 (such as the C-arm instrumentation) can have a known physical transform or relationship with respect to a mounting position of the EM field generator of the EM sensor system 306. Such known relationship can be used to register the fluoroscopy image space to the EM sensor space. The connection 330 represents a mapping or relationship between the fluoroscopy imaging system 312 and an anatomical map depicted by the graphical user interface 314.

In the example of FIG. 3, the CT imaging system 310 and fluoroscopy imaging system 312 are illustrated as separated systems. However, in some other implementations, a single imaging system may perform the functions of both the CT imaging system 310 and fluoroscopy imaging system 312.

The position, shape, or orientation of an instrument, such as an endoscope, can be determined using any one or more of the systems 302-312, which can facilitate generation of graphical interface data representing the estimated position or shape of the instrument relative to an anatomical map depicted by the graphical interface 314. The graphical interface 314 can be displayed on a display device, such as via the control system 118 or robotic system 102, or another device. In some implementations, the graphical interface 314 also may indicate a position of a target within the anatomy that has been designated for treatment.

Although the systems 302-312 have been described in a particular order, the operations or functions associated therewith can be performed in different orders. In some implementations, the systems 302-312 can be used in different ways. In some other implementations, registration can occur between different systems and modalities.

In some aspects, one or more of the systems 302-312 may be used to generate the graphical interface 314 preoperatively or determine a location of one or more targets within an anatomical map depicted by the graphical interface 314 during a preoperative phase of a medical procedure. However, a graphical interface 314 generated using a preoperative CT scan may not accurately reflect the spatial relationship between a medical instrument and a target during an intraoperative phase. For example, changes in the patient's anatomy or the medical environment can cause the spatial relationship between the instrument and the target to deviate from what is depicted in the graphical interface 314. Example factors that may cause such deviations include EM distortion, poor registration (or mapping) between the sensor data and the image data, outdated preoperative scans, and anatomical deformations, among other examples. Thus, in some other aspects, one or more of the systems 302-312 may be used to determine a location of a medical instrument and/or position of a target relative to an anatomical map depicted by the graphical interface 314 during an intraoperative phase of the medical procedure.

A medical system (or a control system associated therewith) may determine the position of a target in relation to an anatomical map by segmenting image data captured via the CT imaging system 310 (such as during a preoperative phase) and/or image data captured via the fluoroscopy imaging system 312 (such as during an intraoperative phase). The accuracy of the spatial information displayed on the graphical interface 314 thus depends, at least in part, on the accuracy of the image segmentation. In some aspects, the medical system may segment the image data using thresholding techniques. For example, the medical system may classify voxels having values above or equal to a threshold intensity as belonging to the target and may classify voxels having values below the threshold intensity as belonging to the background. However, thresholding often provides a relatively coarse approximation of a target's edges or boundaries (often resulting in over-segmentation or under-segmentation). Aspects of the present disclosure recognize that image segmentation can be more accurately performed through machine learning. Thus, in some other aspects, the medical system may segment the image data based at least in part on a machine learning model.

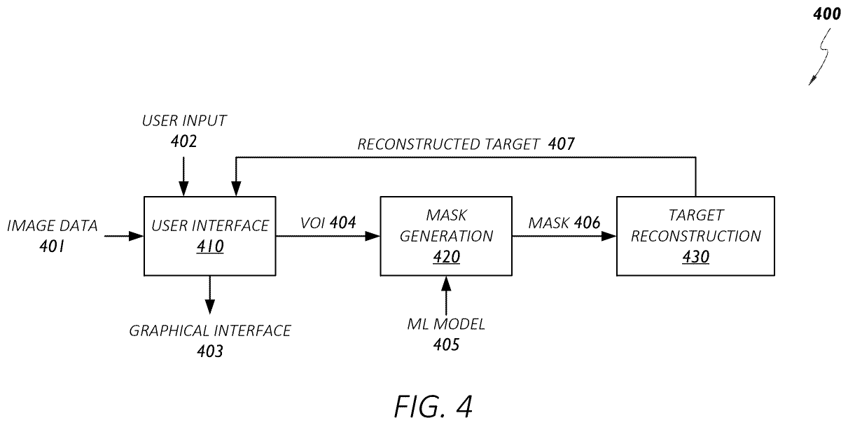

FIG. 4 shows a block diagram of an example segmentation system 400, according to some implementations. In some aspects, the segmentation system 400 may be implemented by a medical system such as the medical system 100 of FIG. 1. With reference to FIG. 2, the segmentation system 400 may be one example of any of the control circuitry 208 or 224. The segmentation system 400 is configured to generate or update a graphical interface 403 that depicts or otherwise indicates a shape, geometry, boundary, eccentricity, texture, and/or position of an object of interest (also referred to herein as a “target”) on an anatomical map. In some implementations, the graphical interface 403 may be one example of the graphical interface 314 of FIG. 3. For example, the graphical interface 403 may further depict a spatial relationship between a medical instrument and the target on the anatomical map.

The segmentation system 400 includes a user interface component 410, a mask generation component 420, and a target reconstruction component 430. The user interface component 410 is configured to generate the graphical interface 403 based at least in part on image data 401 representing a 3D image or model of an anatomy. For example, the image data 401 may include one or more tomograms captured via an imaging system (such as any of the CT imaging system 310 or the fluoroscopy imaging system 312 of FIG. 3). In some aspects, the image data 401 may be acquired during a preoperative phase of a medical procedure. In some other aspects, the image data 401 may be acquired during an intraoperative phase of the medical procedure. In some implementations, the user interface component 410 may use the image data 401 to render an anatomical map on the graphical interface 403 (such as the anatomical map depicted on the graphical interface 314 of FIG. 3).

The user interface component 410 may further provide at least a portion of the image data 401 to the mask generation component 420. In some aspects, the user interface component 410 may crop the image data 401 that is provided to the mask generation component 420 so that the cropped portion of the image data includes only a volume of interest (VOI) 404. In some implementations, the user interface component 410 may determine the VOI 404 based on one or more user inputs 402. For example, a user may select a location of the target on the anatomical map and/or one or more cross-sections of the anatomy, via the user inputs 402, and the user interface component 410 may extract the VOI 404 around the selected location. The user inputs 402 may be provided via any suitable input device (such as touchscreens, touchpads, buttons, switches, mice, keyboards, keypads, joysticks, or scroll wheels, among other examples). In some other implementations, the user interface component 410 may determine the VOI 404 based on one or more image processing operations. For example, the user interface component 410 may analyze the image data 401 using machine learning or statistical analysis techniques to determine an estimated location for the target. In some implementations, the size and/or shape of the VOI 404 may be preconfigured (such as a square or rectangular volume). In some other implementations, the size and/or shape of the VOI 404 may be adjustable by the user (such as to accommodate targets of different sizes).

The mask generation component 420 is configured to produce a segmentation mask 406 based on the VOI 404. The segmentation mask 406 may be a binary mask that delineates the target (also referred to as the “foreground”) from the remainder of the VOI 404 (also referred to as the “background”). For example, the segmentation mask 406 may be a 3D volume having the same dimensions as the VOI 404 and each point or voxel of the segmentation mask 406 may map to a respective voxel of the VOI 404. More specifically, each voxel of the segmentation mask 406 may have a binary value (such as “1” or “0”) indicating whether the corresponding voxel of the VOI 404 belongs to the foreground or the background. In some implementations, the mask generation component 420 may infer the segmentation mask 406 from the VOI 404 using a machine learning (ML) model 405. For example, the machine learning model 405 may be trained to segment a class of objects associated with the target (such as nodules within an anatomy). In some implementations, the mask generation component 420 may deploy the ML model 405 using an artificial intelligence (AI) accelerator, neural processing unit (NPU), or graphics processing unit (GPU) to accelerate the inferences.

The target reconstruction component 430 is configured to generate a 3D model of the target (also referred to as a “reconstructed target”) 407 based on the segmentation mask 406. In some implementations, the target reconstruction component 430 may reconstruct a polygon mesh from the segmentation mask 407 using a mesh generation algorithm (such as the Marching Cubes algorithm) and provide the polygon mesh, as the reconstructed target 407, to the user interface component 410. A polygon mesh is a collection of vertices (points in 3D space), triangles (line segments connecting adjacent vertices), and faces (flat surfaces bounded by edges) that defines the shape or boundaries of a polyhedral object. The user interface component 410 may superimpose or map the reconstructed target 407 onto the anatomical map to model the position and shape of the target in relation to the surrounding anatomy. For example, the user interface component 410 may transform each point of the reconstructed target 407 to a respective point on the anatomical map based on a linear translation that describes the location of the VOI 404 in the associated image space.

In some aspects, the user interface component 410 may determine a position of the target based on the mapping of the reconstructed target 407 to the anatomical map. In some other aspects, the user interface component 410 may estimate a shape or geometry (such as a volume or diameter) of the target based on a respective shape or geometry of the reconstructed target 407. For example, the pose and/or geometry of the target can be used to provide guidance for navigating a medical instrument to the target (such as described with reference to FIGS. 1-3). In some implementations, the user interface component 410 may determine a spatial relationship between an instrument (such as an endoscope) and the target based at least in part on the position of the reconstructed target 407. In some other implementations, the user interface component 410 may confirm tool-in-lesion of an instrument (such as a biopsy needle) based at least in part on the pose and geometry of the reconstructed target 407. Still further, in some implementations, the user interface component 410 may support postoperative procedures and/or treatments that rely on accurate modeling of a target's geometrical properties, mechanical properties, and/or location (such as for planning of drug dosage and/or delivery locations, biopsy patterns and/or locations, and ablation, among other examples).

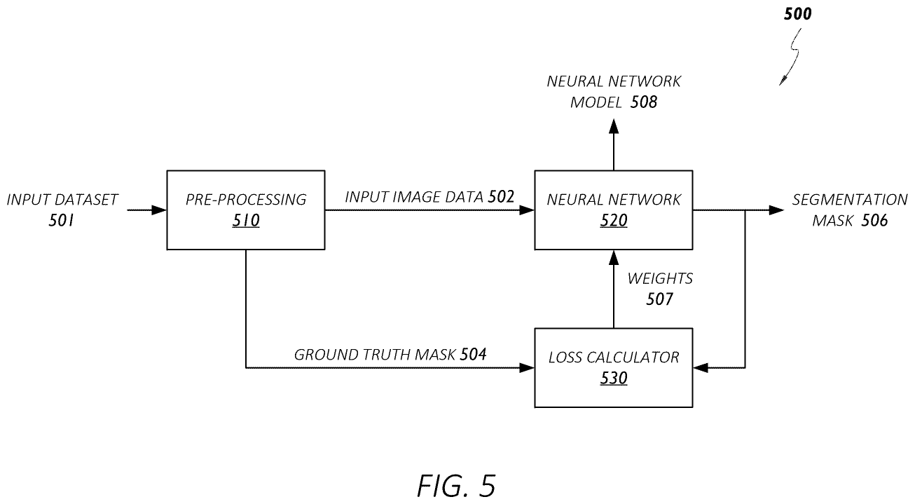

FIG. 5 shows a block diagram of an example machine learning system 500, according to some implementations. The machine learning system 500 is configured to produce a neural network model 508 based, at least in part, on a large volume of input datasets 501 that included annotated images (or tomograms) of an anatomy. In some implementations, the neural network model 508 may be one example of the ML model 405 of FIG. 4. For example, the neural network model 508 may be trained to infer segmentation masks (such as the segmentation mask 406) from tomographic images of an anatomy (such as the VOI 404).

The machine learning system 500 includes a pre-processing component 510, a neural network 520, and a loss calculator 530. The pre-processing component 510 is configured to prepare input image data 502 and ground truth masks 504 based on the input dataset 501. For example, the input image data 502 may depict a target (such as a nodule) within a 3D volume cropped from a larger 3D image of an anatomy (similar to the VOI 404) and the ground truth mask 504 may be a binary mask delineating the target from the background of the input image data 502 (similar to the segmentation mask 406). The ground truth mask 504 may be annotated by assigning a label or class to each point or voxel (such as to indicate whether the voxel belongs to the foreground or the background). In some implementations, the pre-processing component 510 may resample the input image data 502 (and ground truth mask 504) to resize the image or volume provided as input to the neural network 520. In some other implementations, the pre-processing component 510 may normalize a format and/or one or more visual properties (such as a brightness and/or gamma correction) of the input image data 502. With reference for example to FIG. 4, the mask generation component 420 may perform the same pre-processing (such as normalization and/or resampling) on the VOI 404 as the pre-processing component 510. Still further, in some implementations, the pre-processing component 510 may augment the input dataset 501 (for increased diversity) by applying various transformations to at least some of the input image data 502 (such as rotations, scaling, flipping, cropping, or elastic deformations).

The neural network 520 receives the input image data 502 and attempts to recreate the ground truth mask 504. For example, the neural network 520 may form a network of connections across multiple layers of artificial neurons that begin with the input image data 502 and lead to a segmentation mask 506. The connections are weighted (via weights 507) to result in a segmentation mask 506 that closely resembles the ground truth mask 504. In some aspects, the neural network 520 may include a softmax layer to binarize each point or voxel of the segmentation mask 506 (such as by classifying values greater than 0.5 as foreground or “1” and classifying values less than 0.5 as background or “0”). The training operation may be performed over multiple iterations. In each iteration, the neural network 520 produces a respective segmentation mask 506 based on the weighted connections across the layers of artificial neurons, and the loss calculator 530 updates the weights 507 associated with the connections based on an amount of loss (or error) between the segmentation mask 506 and the ground truth mask 504. The neural network 520 may output the weighted connections as the neural network model 508 when certain convergence criteria are met (such as when the loss falls below a threshold level or after a predetermined number of training iterations).

In some implementations, the neural network 520 may have a U-Net architecture. However, various other machine learning architectures also may be implemented by the neural network 520. In some aspects, a portion of the input dataset 501 may be used for training and another portion of the input dataset 501 may be used for validation. The validation dataset can be used to evaluate the segmentation accuracy of the neural network model 508. The accuracy of the neural network model 508 can be measured according to various suitable metrics including, but not limited to, the Dice similarity coefficient (which measures a similarity between a segmentation mask and a ground truth mask). In some implementations, the neural network 520 may self-configure various hyperparameters (such as an optimal number of layers, filter sizes, and/or other architectural parameters) using automated tools.

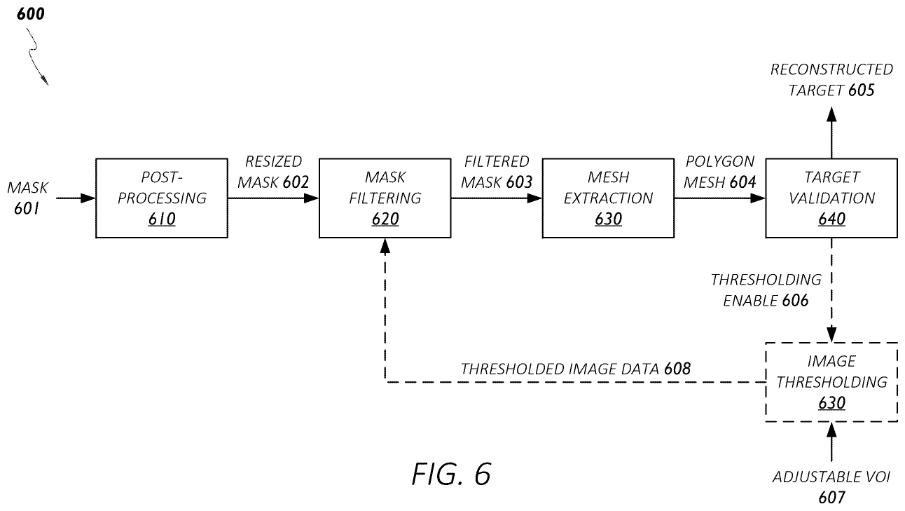

FIG. 6 shows a block diagram of an example target reconstruction system 600, according to some implementations. In some implementations, the target reconstruction system 600 may be one example of the target reconstruction component 430 of FIG. 4. More specifically, the target reconstruction system 600 may be configured to generate a reconstructed target 605 based, at least in part, on a segmentation mask 601. With reference to FIG. 4, the segmentation mask 601 may be one example of the segmentation mask 406 and the reconstructed target 605 may be one example of the reconstructed target 407.

The target reconstruction system 600 includes a post-processing component 610, a mask filtering component 620, and a mesh extraction component 630, and a target validation component 640. The post-processing component 610 is configured to resample (such as by up-sampling or down-sampling) the segmentation mask 601 to produce a resized mask 602 that matches the original resolution of a VOI from which the mask is derived (such as the VOI 404 of FIG. 4). More specifically, the post-processing component 610 may reverse any resampling (or resizing) of the VOI performed by the mask generation component 420 for input to the ML model 405 (such as described with reference to the pre-processing component 520 of FIG. 5). In some aspects, the mask generation component 420 may segment multiple nodules or targets in the same VOI. As a result, the resized mask 602 may include multiple foreground objects.

The mask filtering component 620 is configured to filter or remove any additional foreground objects (other than the target) from the resized mask 602. In other words, the mask filtering component 620 produces a filtered mask 603 that includes only the segmented target from the resized mask 602. In some implementations, the mask filtering component 620 may isolate the target by applying a connectivity filter to the resized mask 602. A connectivity filter is an algorithm or filter that can detect a “connectivity” of various cells or regions in the resized mask 602 and extract a grouping of cells that are connected to one another (such as cells that share common points and/or satisfy other connectivity criteria). In some implementations, the mask filtering component 620 may use the center or origin of the resized mask 602 (which corresponds to the center or origin of the VOI) as a seed for selecting the target to be extracted by the connectivity filter.

The mesh extraction component 630 is configured to generate a polygon mesh based on the filtered mask 603. As described with reference to FIG. 4, a polygon mesh is a collection of vertices, triangles, and faces that defines the shape or boundaries of a polyhedral object. Thus, the polygon mesh 604 may be a 3D model of the target in the filtered mask 603. In some implementations, the mesh extraction component 630 may extract the polygon mesh 604 from the filtered mask 603 using a mesh generation algorithm (such as the Marching Cubes algorithm). However, aspects of the present disclosure recognize that, in some instances, an ML model (such as the ML model 405 of FIG. 4 or the neural network model 508 of FIG. 5) may fail to segment the target from the VOI. In such instances, the segmentation mask 601 may not accurately delineate the target. As a result, the polygon mesh 604 may not be suitable for modeling the reconstructed target 605.

The target validation component 640 is configured to selectively output the polygon mesh 604 as the reconstructed target 605. In some aspects, the target validation component 640 may determine whether the polygon mesh 604 depicts a valid 3D model of the target based on a number of vertices in the mesh. For example, if the number of vertices is equal to zero (due to failed segmentation by the ML model), the target validation component 640 may determine that the polygon mesh 604 does not depict a valid 3D model of the target. In some implementations, the target validation component 640 may output the polygon mesh 604 as the reconstructed target 605 in response to determining that the polygon mesh 604 depicts a valid 3D model of the target. In some other implementations, the target validation component 640 may cause the target to be segmented by alternative means (other than the ML model) in response to determining that the polygon mesh 604 does not depict a valid 3D model of the target.

In some aspects, the target validation component 640 may assert or output a thresholding enable signal 606 to an image thresholding component 630 in response to determining that the polygon mesh 604 does not depict a valid 3D model of the target. The image thresholding component 630 is configured to extract thresholded image data 608 from an adjustable VOI 607 in response to receiving or detecting the thresholding enable signal 606. The location and/or dimensions of the adjustable VOI 607 can be provided or otherwise specified based on user input (such as via bounding boxes). In some implementations, the image thresholding component 630 may extract the thresholded image data 608 using one or more image thresholding techniques. For example, the image threshold component 630 may compare each voxel of the adjustable VOI 607 to a threshold intensity or value (associated with a target) and classify each voxel as belonging to the target or a background based whether the value of the voxel is higher or lower than the threshold value. Thus, the resulting thresholded image data 608 may be a binary mask similar to the resized mask 602.

In some implementations, the thresholded image data 608 may be processed through the mask filtering component 620 in a manner similar to the resized mask 602. For example, the mask filtering component 620 may extract a filtered mask 630 from the thresholded image data 608 using a connectivity filter. The resulting filtered mask 603 may be converted to a polygon mesh 604, via the mesh extraction component 630, and subsequently output by the target validation component 640 as the reconstructed target 605. In the example of FIG. 6, the connectivity filter is shown to be applied on a segmentation mask (such as the resized mask 602) prior to generating a polygon mesh (such as the polygon mesh 604). However, in some other implementations, the connectivity filter may be applied to the polygon mesh rather than the segmentation mask. In such implementations, the polygon mesh may include multiple targets or nodules prior to applying the connectivity filter.

As described with reference to FIG. 6, the mask filtering component 620 uses the center or origin of the resized mask 602 (which corresponds to the center origin of the VOI) as a seed for resolving multiple segmented objects. In some implementations, the center or origin of the VOI may coincide with a user-selected point (also referred to as a “seed location”) on a 3D image of an anatomy from which the VOI is extracted (such as based on the user input 402 of FIG. 4). In some other implementations, the seed location may be programmatically selected based on one or more image processing operations (such as machine learning or statistical analysis). However, a VOI may become truncated if the selected point is too close to an edge or boundary of the 3D image (because the VOI cannot extend beyond the parameters of the 3D image). As a result, the center or origin of the truncated VOI may be offset relative to the user-selected point. In some aspects, a medical system may add one or more padding bits to a truncated VOI so that the center or origin of the resulting volume tracks the selected point that is used as a seed for the connectivity filter.

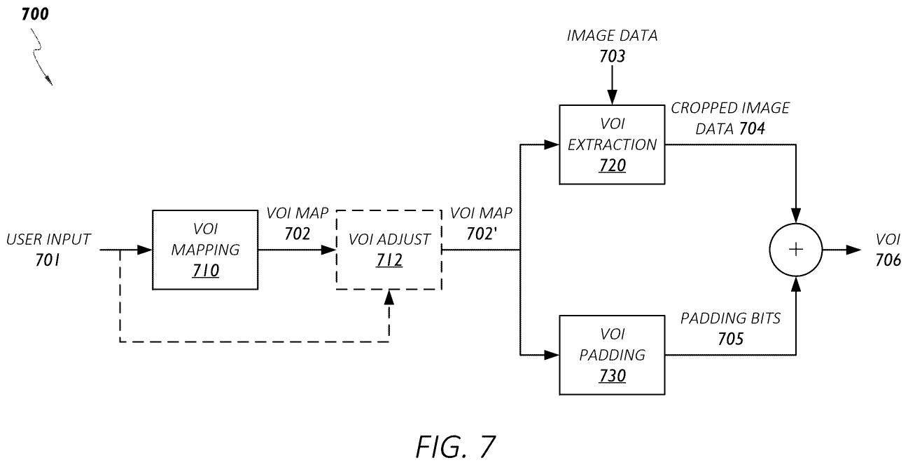

FIG. 7 shows a block diagram of an example image cropping system 700, according to some implementations. In some implementations, the image cropping system 700 may be one example of the user interface component 410 of FIG. 4. More specifically, the image cropping system 700 is configured to crop a set of image data 703 depicting a 3D image of an anatomy so that the resulting cropped image data includes only a VOI 706 associated with a user input 701. With reference to FIG. 4, the user input 701 may be one example of the user input 402, the image data 703 may be one example of the image data 401, and the VOI 706 may be one example of the VOI 404.

The image cropping system 700 includes a VOI mapping component 710, a VOI extraction component 720, and a VOI padding component 730. The VOI mapping component 710 is configured to produce a VOI map 702 based on the user input 701. The VOI map 702 represents a mapping of a 3D volume (such as a square or rectangle) to an image space associated with the image data 703. In some aspects, a user may select a seed location on the 3D image and/or one or more cross-sections of the anatomy. In some other aspects, the VOI mapping component 710 may determine the seed location based on one or more image processing operations (such as machine learning or statistical analysis). The VOI mapping component 710 may project the VOI map 702 onto the image space so that the center or origin of the VOI map 702 coincides with the selected seed location. In some implementations, the VOI map 702 may have a predetermined size and/or dimensions. In some other implementations, the size and/or dimensions of the VOI map 702 may be adjustable through a VOI adjustment component 712. For example, the VOI adjustment component 712 may alter the size and/or dimensions of the VOI map 702, to produce an adjusted VOI map 702′, based on user input 701.

The VOI extraction component 720 is configured to crop the image data 703 based on the VOI map 702 (or the adjusted VOI map 702′) to produce cropped image data 704 that is bounded by the dimensions of the VOI map 702 or 702′. In other words, the cropped image data 704 may include only the voxels of the image data 703 that fall within the 3D volume defined by the VOI map 702 or 702′. In some implementations, the cropped image data 704 may be output by the image cropping system 700 as the VOI 706. However, as described with reference to FIG. 6, the dimensions of the cropped image data 704 may be truncated compared to the dimensions of the VOI map 702 or 702′ if the seed location is too close to an edge or boundary of the 3D image. As a result, the center or origin of a volume defined by the cropped image data 704 may be offset relative to the center or origin of the VOI map 702 or 702′. Because the center or origin of the VOI 706 is used as a seed for resolving multiple segmented objects (such as described with reference to FIG. 6), truncating or changing the dimensions of the VOI 706 may result incorrect application of the connectivity filter.

The VOI padding component 730 is configured to selectively output one or more padding bits 705 based on the location and dimensions of the VOI map 702 (or the adjusted VOI map 702′). More specifically, the VOI padding component 730 may use the padding bits 705 to pad the cropped image data 704 so that the dimensions of the resulting VOI 706 match the dimensions of the VOI map 702 or 702′. For example, the VOI padding component 730 may determine whether any portions of the VOI map 702 or 702′ extend beyond one or more edges or boundaries of the 3D image and may pad such portions of the VOI map 702 or 702′ with one or more padding bits 705. Example suitable padding techniques include zero padding and mirror padding (also referred to as “symmetric padding”), among other examples. In some implementations, each of the padding bits 705 may have a negative value (such as −1000). Any padding bits 705 output by the VOI padding component 730 are combined with the cropped image data 704 to produce the VOI 706. As a result, the padding bits 705 may ensure that the seed location coincides with the center or origin of the VOI 706.

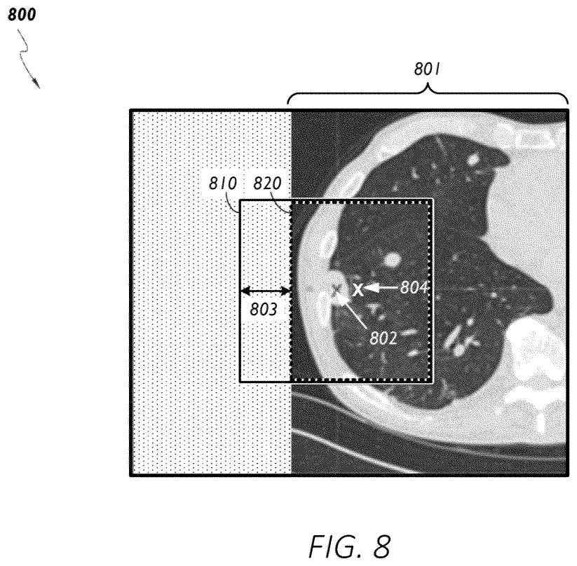

FIG. 8 shows an example image of an anatomy with VOI padding 800, according to some implementations. More specifically, FIG. 8 depicts a cross-section 801 of the anatomy and a VOI map 810 associated therewith. In some implementations, the VOI map 810 may be used by an image cropping system (such as the image cropping system 700 of FIG. 7) to crop a VOI from the image cross-section 801 (such as the VOI 706 of FIG. 7). With reference to FIG. 7, the VOI map 810 may be one example of the VOI map 702 (or the adjusted VOI map 702′) and the image cross-section 801 may be an example slice of the image data 703.

As shown in FIG. 8, the VOI map 810 is centered around a seed location 802 (labeled with a dark “X”) that is aligned with a target or nodule in the anatomy. However, due to the proximity of the seed location 802 to the left-most edge of the image cross-section 801, a portion 803 of the VOI map 810 extends beyond the boundary of the cross-section 801. As a result, the image cropping system may crop a region 820 of the image cross-section 801 that is smaller than the dimensions of the VOI map 810. With reference to FIG. 7, the cropped region 820 may be one example of the cropped image data 704. Due to the reduction in dimensionality, the cropped portion 820 has a center or origin 804 (labeled with a lighter “X”) that is offset relative to the seed location 802. As shown in FIG. 8, the center 804 of the cropped portion 820 is not aligned with the target or nodule in the anatomy. To prevent the center of the VOI from shifting to the center 804 of the cropped region 820, the image cropping system may pad the portion 803 of the VOI map 810 with one or more padding bits (such as the padding bits 705 of FIG. 7). As a result of such padding, the VOI output by the image cropping system will have the dimensions as the VOI map 810.

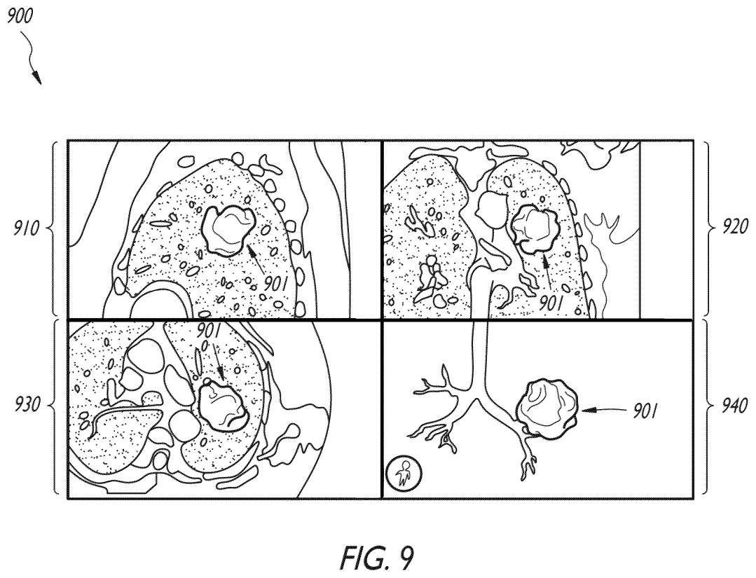

FIG. 9 shows an example graphical interface 900 depicting a reconstructed nodule 901 superimposed on images of an anatomy, according to some implementations. In some implementations, the graphical interface 900 may be one example of the graphical interface 403 of FIG. 4. With reference to FIG. 4, the reconstructed nodule 901 may be one example of the reconstructed target 407.

The graphical interface 900 is subdivided into four display regions 910-940, where each of the first three display regions 910-930 is shown to display a respective cross-sectional view of the anatomy (including transverse, coronal, and sagittal cross-sections) and the fourth display region 940 is shown to display a volumetric view of the anatomy. As shown in FIG. 9, the reconstructed nodule 901 is a 3D model of a nodule having clearly defined edges and boundaries that model the actual boundaries of the nodule. As such, the reconstructed nodule 901 can be used to facilitate various types of treatments and/or medical procedures including instrument navigation, tool-in-lesion confirmation, planning drug dosage and/or delivery locations, planning biopsy patterns and/or locations, and ablation, among other examples (such as described with reference to FIGS. 1-4). More specifically, the reconstructed nodule 901 can provide a precise and accurate representation of the position and geometry of the nodule during a pre-operative phase, an intra-operative phase, and/or a post-operative phase of a medical procedure.

The graphical interface 900 of FIG. 9 is merely shown for purposes of example. In actual implementations, various other graphical interfaces can be generated using the reconstructed nodule 901. Additionally, various aspects of the graphical interface 900 may be customized to user preferences. Example suitable variations and/or customization options may include, among other examples, changing the number or locations of the cross-section views and/or the volumetric view, changing the relative dimensions of one or more views, displaying one or more oblique views (which may not be orthogonal to the transverse, coronal, or sagittal planes), adjusting one or more rendering parameters (such as color, opacity, or intensity of various features) of the volumetric view (such as via a transfer function editor), or changing a color used to highlight one or more display regions 910-940 of the graphical interface 900. The graphical interface 900 also may include various text, fonts, shapes, buttons, icons, and/or other graphical features not shown in FIG. 9.

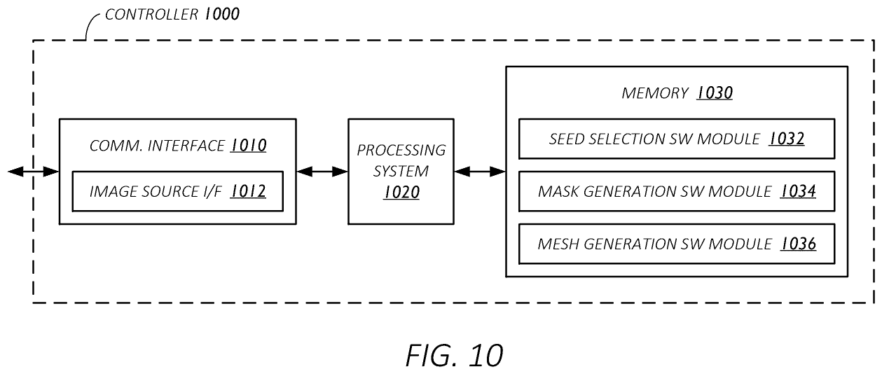

FIG. 10 shows a block diagram of an example controller 1000 for a medical system, according to some implementations. In some implementations, the controller 1000 may be one example of the segmentation system 400 of FIG. 4. More specifically, the controller 1000 is configured to analyze a target within an anatomy.

The controller 1000 includes a communication interface 1010, a processing system 1020, and a memory 1030. The communication interface 1010 is configured to communicate with one or more components of the medical system. More specifically, the communication interface 1010 includes an image source interface (I/F) 1012 for communicating with one or more image sources (such as the CT imaging system 310 and/or the fluoroscopy imaging system 312 of FIG. 3). In some implementations, the image source interface 1012 may receive image data representing a 3D image of the anatomy.

The memory 1030 may include a non-transitory computer-readable medium (including one or more nonvolatile memory elements, such as EPROM, EEPROM, Flash memory, or a hard drive, among other examples) that may store the following software (SW) modules: a seed selection SW module 1032 to select a seed location for the target in the 3D image of the anatomy; a mask generation SW module 1034 to infer a segmentation mask associated with the seed location from at least a portion of the received image data based on a neural network model trained to segment a class of objects associated with the target; and a mesh generation SW module 1036 to generate a polygon mesh representing a geometry of the target based on the segmentation mask. Each of the software modules 1032-1036 includes instructions that, when executed by the processing system 1020, causes the controller 1000 to perform the corresponding functions.

The processing system 1020 may include any suitable one or more processors capable of executing scripts or instructions of one or more software programs stored in the controller 1000 (such as in the memory 1030). For example, the processing system 1020 may execute the seed selection SW module 1032 to select a seed location for the target in the 3D image of the anatomy. The processing system 1020 also may execute the mask generation SW module 1034 to infer a segmentation mask associated with the seed location from at least a portion of the received image data based on a neural network model trained to segment a class of objects associated with the target. The processing system 1020 may further execute the mesh generation SW module 1036 to generate a polygon mesh representing a geometry of the target based on the segmentation mask.

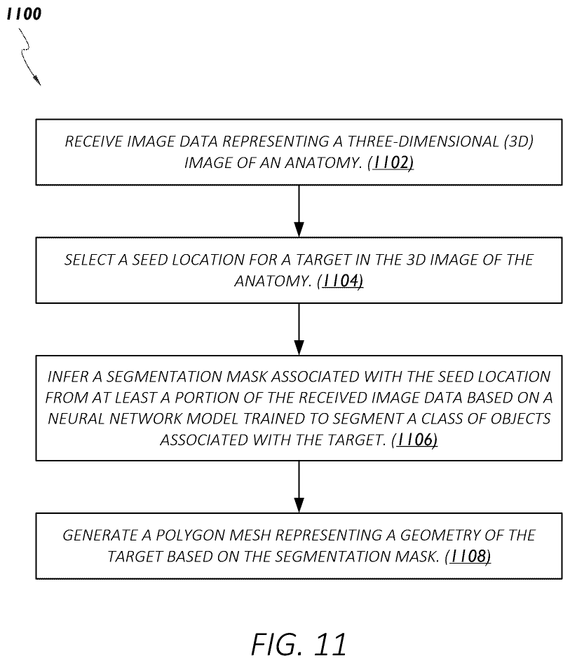

FIG. 11 shows an illustrative flowchart depicting an example operation 1100 for navigating an instrument within an object, according to some implementations. In some implementations, the example operation 1100 may be performed by a controller for a medical system such as the controller 1000 of FIG. 10 or the segmentation system 400 of FIG. 4.

The controller receives image data representing a 3D image of an anatomy (1102). The controller selects a seed location for a target in the 3D image of the anatomy (1104). In some implementations, the controller may receive user input indicating the seed location. In some other implementations, the controller may determine the seed location based on one or more image processing operations. The controller infers a segmentation mask associated with the seed location from at least a portion of the received image data based on a neural network model trained to segment a class of objects associated with the target (1106). The controller further generates a polygon mesh representing a geometry of the target based on the segmentation mask (1108).

In some aspects, the inferring of the segmentation mask may include mapping a volume of interest (VOI) to the 3D image based on the seed location and cropping the 3D image based on the VOI so that the segmentation mask is inferred from voxels of the 3D image that are bounded by the VOI. In some implementations, the inferring of the segmentation mask may further include resizing the cropped 3D image for input to the neural network model and resizing the segmentation mask based on dimensions of the VOI. In some implementations, the VOI may be centered at the seed location. In some implementations, the controller may further pad the received image data with one or more padding bits associated with a region of the VOI that exceeds a boundary of the 3D image.

In some aspects, the controller may determine a spatial relationship between an instrument and the target based at least in part on the polygon mesh. In some implementations, the controller may further superimpose the polygon mesh on the 3D image and generate a graphical interface depicting the spatial relationship between the instrument and the target based on the 3D image having the polygon mesh superimposed thereon. In some implementations, the polygon mesh may be superimposed on the 3D image based on the mapping of the VOI to the 3D image. In some implementations, the determining of the spatial relationship may further include determining a position of the target in a coordinate space associated with the image data based on a position of the polygon mesh in relation to the 3D image.

In some aspects, the determining of the spatial relationship may further include calculating a number of vertices in the polygon mesh, determining whether the polygon mesh represents a reconstruction of the target based on the calculated number of vertices, comparing each voxel within a portion of the 3D image to a threshold value responsive to determining that the polygon mesh does not represent a reconstruction of the target, and determining the spatial relationship between the instrument and the target based on comparing each voxel within the portion of the 3D image to the threshold value. In some other aspects, the controller may further estimate a volume or diameter of the target based on the polygon mesh.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A method for analyzing a target within an anatomy, comprising:
receiving image data representing a three-dimensional (3D) image of the anatomy;
selecting a seed location for the target in the 3D image of the anatomy;
inferring a segmentation mask associated with the seed location from at least a portion of the received image data based on a neural network model trained to segment a class of objects associated with the target; and
generating a polygon mesh representing a geometry of the target based on the segmentation mask.

2. The method of claim 1, wherein the selecting of the seed location comprises receiving user input indicating the seed location.

3. The method of claim 1, wherein the selecting of the seed location comprises determining the seed location based on one or more image processing operations.

4. The method of claim 1, wherein the inferring of the segmentation mask comprises:
mapping a volume of interest (VOI) to the 3D image based on the seed location; and
cropping the 3D image based on the VOI so that the segmentation mask is inferred from voxels of the 3D image that are bounded by the VOI.

5. The method of claim 4, wherein the inferring of the segmentation mask further comprises:
resizing the cropped 3D image for input to the neural network model; and
resizing the segmentation mask based on dimensions of the VOI.

6. The method of claim 4, wherein the VOI is centered at the seed location, the method further comprising:
padding the received image data with one or more padding bits associated with a region of the VOI that exceeds a boundary of the 3D image.

7. The method of claim 4, further comprising:
determining a spatial relationship between an instrument and the target based at least in part on the polygon mesh.

8. The method of claim 7, further comprising:
superimposing the polygon mesh on the 3D image; and
generating a graphical interface depicting the spatial relationship between the instrument and the target based on the 3D image having the polygon mesh superimposed thereon.

9. The method of claim 8, wherein the polygon mesh is superimposed on the 3D image based on the mapping of the VOI to the 3D image.

10. The method of claim 8, wherein the determining of the spatial relationship further comprises determining a position of the target in a coordinate space associated with the image data based on a position of the polygon mesh in relation to the 3D image.

11. The method of claim 1, wherein the determining of the spatial relationship further comprises:
calculating a number of vertices in the polygon mesh;
determining whether the polygon mesh represents a reconstruction of the target based on the calculated number of vertices;
comparing each voxel within a portion of the 3D image to a threshold value responsive to determining that the polygon mesh does not represent a reconstruction of the target; and
determining the spatial relationship between the instrument and the target based on comparing each voxel within the portion of the 3D image to the threshold value.

12. The method of claim 1, further comprising:
estimating a volume or diameter of the target based on the polygon mesh.

13. A controller for a medical system, comprising:
a processing system; and
a memory storing instructions that, when executed by the processing system, cause the controller to:
receive image data representing a three-dimensional (3D) image of the anatomy;
select a seed location for a target in the 3D image of the anatomy;
infer a segmentation mask associated with the seed location from at least a portion of the received image data based on a neural network model trained to segment a class of objects associated with the target; and
generate a polygon mesh representing a geometry of the target based on the segmentation mask.

14. The controller of claim 13, wherein the selecting of the seed location comprises determining the seed location based on one or more image processing operations.

15. The controller of claim 13, wherein the inferring of the segmentation mask comprises:
mapping a volume of interest (VOI) to the 3D image based on the seed location;
cropping the 3D image based on the VOI so that the segmentation mask is inferred from voxels of the 3D image that are bounded by the VOI;
resizing the cropped 3D image for input to the neural network model; and
resizing the segmentation mask based on dimensions of the VOI.

16. The controller of claim 15, wherein the VOI is centered at the seed location, execution of the instructions further causing the controller to:
pad the received image data with one or more padding bits associated with a region of the VOI that exceeds a boundary of the 3D image.

17. The controller of claim 15, wherein execution of the instructions further causes the controller to:
determine a spatial relationship between an instrument and the target based at least in part on the polygon mesh.

18. The controller of claim 17, wherein execution of the instructions further causes the controller to:
superimpose the polygon mesh on the 3D image based on the mapping of the VOI to the 3D image; and
generate a graphical interface depicting the spatial relationship between the instrument and the target based on the 3D image having the polygon mesh superimposed thereon.

19. The controller of claim 13, wherein the determining of the spatial relationship further comprises:
calculating a number of vertices in the polygon mesh;
determining whether the polygon mesh represents a reconstruction of the target based on the calculated number of vertices;
comparing each voxel within a portion of the 3D image to a threshold value responsive to determining that the polygon mesh does not represent a reconstruction of the target; and
determining the spatial relationship between the instrument and the target based on comparing each voxel within the portion of the 3D image to the threshold value.

20. The controller of claim 13, wherein execution of the instructions further causes the controller to:
estimate a volume or diameter of the target based on the polygon mesh.
